(19) United States
(12) Patent Application Publication
(10) Pub. No.: US 2026/0354416 A1
(43) Pub. Date: Nov. 12, 2026

(54) A COMPOSITION FOR MODULATING GROWTH OF MICROORGANISMS

(71) Applicant: DSM IP Assets B.V., Maastricht (NL)

(72) Inventors: Mathias SPIEGELHOFER, Kaiseraugst (CH); Martin PFEFFER, Kaiseraugst (CH)

(21) Appl. No.: 19/131,724

(22) PCT Filed: Nov. 15, 2023
(86) PCT No.: PCT/EP2023/081899

§ 371 Date: May 21, 2025

(30) Foreign Application Priority Data
Nov. 30, 2022 (EP) 22210509.0

(51) Int. Cl. A23K 20/111; A23K 20/105

(52) U.S. Cl. CPC A23K 20/111; A23K 20/105

(57) ABSTRACT

The present invention provides a composition for modulating growth of microorganisms, comprising at least one additional organic acid and/or at least one salt thereof, and benzoic acid and/or at least one salt thereof, and use thereof.

### TECHNICAL FIELD

The present invention relates to a composition for modulating growth of microorganisms and use thereof.

### BACKGROUND OF THE INVENTION

It is well known in the field of microbiology that the growth behavior of microorganisms depends on environmental conditions. Certain conditions may favor microbial growth while other conditions may reduce growth. Notably, certain environmental conditions may result in a growth reduction or even growth stagnation of some microorganisms, whereas other microorganisms may not be affected at all or may even show increased growth upon exposure to the same conditions.

In practice, environmental conditions are purposefully adapted by humans to either favor or disfavor microbial growth. For instance, efficient growth of selected suitable microorganisms such as bacteria or yeasts or fungi is desired in the feed industry, i.e. by the usage of silage inoculants or in the milk processing industry e.g. to produce yogurt or cheese. Further examples where a controlled increase of microbial growth is favored include fermentation processes such as beer brewing or wine making.

On the other hand, attempts are made to avoid or at least delay spoilage of nutritional products such as food, feedstock or feed due to uncontrolled growth of undesired microorganisms. In this regard, temperature conditions may be chosen to disfavor microbial growth. Typically, products subjected to a risk of microbial spoilage may be kept frozen or at least may be stored at low temperatures, e.g. below 10 °C. Further options to modulate or control microbial growth include the removal of water e.g. by lyophilization, smoking, salting, curing, sugaring or pickling. In some preservation processes, an acidic pH environment is established to disfavor the growth of unfavorable and potentially pathogenic microorganisms.

In this regard, acidifier products including organic acids may be used to maintain food and feed hygiene and prevent spoilage by microorganisms such as bacteria or molds. Organic acids e.g. formic acid, propionic acid or acetic acid as described in EP 2 642 874 may be used to control microbial growth in foodstuff, food or feed with the aim to minimize the risk for foodborne diseases. To this end, acidifier products may be added pre- and/or post-production of finished feed. Additionally, acidifier products may be used to achieve a reduced growth of potentially pathogenic microorganisms in the gastrointestinal tract, but also to improve feed conversion rate and weight gain upon ingestion.

Notwithstanding the above, there is a constant need to improve said acidifier products, e.g. by improving the efficiency of the employed acids. It has now been found surprisingly that benzoic acid and/or at least one salt thereof, in combination with at least one additional organic acid and/or at least one salt thereof, provides enhanced antimicrobial effect.

### SUMMARY OF THE INVENTION

The present invention provides a composition for modulating growth of microorganisms, comprising a) benzoic acid and/or at least one salt thereof, and b) at least one additional organic acid and/or at least one salt thereof.

The present invention also provides use of the above composition for modulating growth of microorganisms, and a method by using the composition for modulating growth of microorganisms.

The present invention further provides use of at least one additional organic acid and/or at least one salt thereof in the composition as defined herein for boosting the effect of benzoic acid and/or at least one salt thereof in modulating growth of microorganisms.

### DETAILED DESCRIPTION OF THE INVENTION

In the first aspect, the present invention provides a composition for modulating growth of microorganisms, wherein the composition comprises a) benzoic acid and/or at least one salt thereof, and b) at least one additional organic acid and/or at least one salt thereof.

In the present invention, the at least one salt of benzoic acid may be selected from the group consisting of sodium benzoate, magnesium benzoate, manganese benzoate, potassium hypophosphite, aluminium benzoate, calcium benzoate and ferric benzoate, preferably sodium benzoate, magnesium benzoate, manganese benzoate and potassium benzoate, and more preferably sodium benzoate.

In the present invention, the at least one additional organic acid is any organic acid other than benzoic acid, which may be selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and/or keto carboxylic acids. Merely for clarification, examples of the short monocarboxylic acids having between 1 and 6 carbon atom(s) are formic acid, acetic acid, propionic acid, butyric acid, isobutyric acid, 3-methylbutyric acid, 2-methylbutyric acid, 2-ethylbutyric acid, valeric acid and hexanoic acid. Examples of the saturated dicarboxylic acids are adipic acid and succinic acid. An example of the unsaturated dicarboxylic acid is fumaric acid. Examples of the unsaturated carboxylic acids are sorbic acid and oleic acid. Examples of the saturated carboxylic acids are stearic acid, octanoic acid (also referred to as caprylic acid), decanoic acid (also referred to as capric acid) and dodecanoic acid (also referred to as lauric acid). Examples of the hydroxycarboxylic acids are lactic acid, malic acid (D-, or L-, or D/L-malic acid), citric acid and tartaric acid. An example of the aromatic carboxylic acids is cinnamic acid. An example of the keto carboxylic acid is pyruvic acid.

Preferably, the at least one additional organic acid is selected from the group consisting of formic acid, acetic acid, propionic acid, butyric acid, isobutyric acid, 3-methylbutyric acid, 2-methylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, cinnamic acid, pyruvic acid, gluconic acid, suberic acid, malonic acid, tannic acid, caffeic acid, ellagic acid, perillic acid and gallic acid.

More preferably, the at least one additional organic acid is selected from formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, cinnamic acid and pyruvic acid, or at least one salt thereof.

Further preferably, the at least one additional organic acid is selected from formic acid, acetic acid, propionic acid, fumaric acid and succinic acid. The most preferably, the at least one additional organic acid is selected from fumaric acid and succinic acid.

In the present invention, the at least one salt of the additional organic acid may be any one of metal salts such as potassium, sodium or calcium salts, and ammonium salts. Examples of the salts of the organic acid include but are not limited to ammonium formate, potassium diformate, sodium diacetate, calcium acetate, ammonium propionate, sodium propionate, calcium propionate, calcium lactate, potassium sorbate, sodium formate, calcium formate, sodium butyrate, sodium sorbate, potassium citrate, sodium citrate and calcium citrate.

In some embodiments, the composition according to the invention may comprise benzoic acid and/or at least one salt thereof in combination with more than one additional organic acid and/or at least one salt thereof. For example, such a composition may comprise benzoic acid and/or at least one salt thereof in combination with two, three, four, five or even more additional organic acids. In preferable embodiments, the composition comprises benzoic acid and/or a salt of benzoic acid and at least three additional organic acids, especially formic acid, acetic acid and propionic acid, wherein the salt of benzoic acid is selected from the group consisting of sodium benzoate, manganese benzoate, magnesium benzoate and potassium benzoate, preferably sodium benzoate or manganese benzoate, more preferably sodium benzoate. In more preferable embodiments, the composition according to the invention comprises benzoic acid and/or a salt of benzoic acid and at least four additional organic acids, especially formic acid, acetic acid, propionic acid and fumaric acid, wherein the salt of benzoic acid is selected from the group consisting of sodium benzoate, manganese benzoate, magnesium benzoate and potassium benzoate, preferably sodium benzoate or manganese benzoate, more preferably sodium benzoate. In further preferable embodiments, the composition according to the invention comprises benzoic acid and/or a salt of benzoic acid and at least five additional organic acids, especially formic acid, acetic acid, propionic acid, fumaric acid and succinic acid, wherein the salt of benzoic acid is selected from the group consisting of sodium benzoate, manganese benzoate, magnesium benzoate and potassium benzoate, preferably sodium benzoate or manganese benzoate, more preferably sodium benzoate.

The composition of the present application may be formulated in the form of a feed additive (premix) or a food additive or a feed or a food for administering to animals. Accordingly, Such a composition may further comprise components, such as one or more of the following: Fat- or water-soluble vitamin(s), trace- and/or macro-mineral(s), (proteinogenic) amino acid(s), organic and/or inorganic adsorbant(s), polyunsaturated fatty acid(s), antimicrobial polypeptide(s), pre- and/or pro-biotic(s), flavoring and/or coloring agent(s), microorganism(s) and/or enzyme(s) to facilitate better nutrient availability and/or to mitigate undesired effect(s) of feed/food contaminant(s).

Such a composition according to the invention may be provided in a form wherein one or more or all of the components is/are provided in solid form (e.g. salt, powder, granulate, pellet etc.) or in liquid form (e.g. aqueous, gel, viscous). It is also considered that the composition may be provided in a manner, wherein one component (e.g. one or more organic acid(s)) is provided in liquid form and a second component is provided in solid form (e.g. benzoic acid), and the composition is formed by combining the two components in a mixture or in a separate form.

According to the present invention, said composition may be provided in a particular manner, wherein the molar ratio of the at least one additional organic acid and/or the at least one salt thereof to benzoic acid and/or the at least one salt thereof is from 0.001:1 to 500:1, preferably from 0.005:1 to 300:1, more preferably from 0.01:1 to 200:1, such as from 0.01:1 to 0.15:1, from 0.02:1 to 0.1:1, from 0.04:1 to 100:1, from 0.05:1 to 100:1, from 0.1:1 to 100:1, from 1:1 to 100:1, from 10:1 to 80:1 and from 20:1 to 40:1. For instance, the at least one additional organic acid and/or the at least one salt thereof may be provided at any concentration from 0.2 mmol to 100 mmol such as 0.2. 0.4, 0.8. 1.5, 3.1, 6.25, 12.5, 25.0, 50.0 and 100 mmol, in combination with benzoic acid and/or at least one salt thereof at any concentration from 0.2 mmol to 20 mmol such as 0.3, 0.6, 1.3, 2.5, 5.0, 10 and 20 mmol, per 1 Kg of the composition in solid form or per 1 L of the composition in liquid form. Preferably, in the composition of the present invention, the molar ratio of the at least one additional organic acid and/or the at least one salt thereof to benzoic acid and/or the at least one salt thereof is in the range of from 0.001:1 to 0.15:1.

The composition of the present invention can be used for modulating growth of microorganisms, i.e., inhibiting the growth of pathogenic microorganisms and/or enhancing the growth of beneficial (i.e. probiotic) microorganisms.

In the present invention, the microorganisms may be any one or more of the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, Lactobacillus, Bifidobacterium, Saccharomyces, Bacillus, Pediococcus, Enterococcus, Streptococcus, Propionibacterium, Pseudomonas and Citrobacter. Particularly, the microorganisms in the present invention may be any one or more of Escherichia coli, Salmonella enterica, Shigella sonnei, Clostridium perfringens, Lactobacillus reuteri, Enterococcus faecium and Bacillus subtilis.

Particularly, the pathogenic microorganisms in the present invention may be at least one of the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella and Tenacibaculum, in particular may be selected from the group consisting of Escherichia coli, Salmonella enterica, Shigella sonnei and Clostridium perfringens.

Particularly, the beneficial microorganisms in the present invention may be at least one microorganism selected from the genera of Lactobacillus, Bifidobacterium, Saccharomyces, Bacillus, Pediococcus, Enterococcus, Streptococcus, Propionibacterium, Pseudomonas and Citrobacter, in particular may be selected from the group consisting of Lactobacillus reuteri, Enterococcus faecium and Bacillus subtilis.

It was unexpectedly found that the composition of the present invention was capable of modulating growth of microorganisms in a synergistic model. In the present invention, the term “in a synergistic model” means that, in a test of modulating growth of microorganisms, for example, according to the example 1 of the present invention, the composition of the present invention provides observed value (“0”) beyond the expected value (“E”) resulting from the individual ingredient of the composition, i.e, benzoic acid and/or salt thereof and the additional organic acid and/or salt thereof. In the present invention, the expected value (“E”) can be calculated according to the formula developed by Colby et al. (1967. Weeds 15(1): 20-22), which is incorporated herein as reference. In the present invention, the expression “observed value (“0”) beyond the expected value (“E”)” means that the result of the observed value minus the expected value, i.e., E-O, is bigger than zero (0), preferably no less than 1, 2, 3, 5, 8 10, and more preferably no less than 10.

Accordingly, the present invention also provides a composition as described above, wherein in a test of modulating growth of microorganisms the composition of the present invention provides observed value (“O”) beyond the expected value (“E”) resulting from the individual ingredient of the composition, i.e, benzoic acid and/or salt thereof and the additional organic acid and/or salt thereof.

In the present invention, the composition may comprise benzoic acid and/or at least one salt thereof and the at least one additional organic acid and/or at least one salt thereof as the only active ingredients for modulating growth of microorganisms. However, the composition of the present invention, besides benzoic acid and/or at least one salt thereof and the at least one additional organic acid and/or at least one salt thereof, may comprises additional active ingredients such as enzymes for modulating growth of microorganisms.

In the second aspect, the present invention provides use of the composition as described herein for modulating growth of microorganisms, i.e., inhibiting the growth of pathogenic microorganisms and/or enhancing the growth of beneficial (i.e. probiotic) microorganisms.

As understood by any person skilled in the art, the microorganisms may be present in a food and/or a feed, and/or in the gastrointestinal tracts of animals. In some embodiments, the present invention provides use of the composition as described herein for modulating growth of microorganisms, especially for inhibiting the growth of pathogenic microorganisms, in a food and/or a feed. In some embodiments, the present invention provides use of the composition as described herein for modulating growth of microorganisms in the gastrointestinal tract, especially in the intestinal tracts (e.g. stomach, duodenum, jejunum, illeum, cecum, and/or large intestine) of animals. In some preferable embodiment, the present invention provides use of the composition as described herein for improving gut health of animals.

In some special embodiments, the present invention provides use of the composition as described herein for the treatment, amelioration and/or prevention of post-weaning diarrhea (PWD) of animals, such as pigs. PWD is often caused by strains of Escherichia coli in the gastrointestinal tract of animals (e.g. Fairbrother et al. 2005. Anim Health Res Rev. 6(1):17-39), resulting in considerable economic loss and animal malady. Notably, growth of such strains can be reduced in the gastrointestinal tract of animals (e.g. pigs) by administering to the animals the composition described herein, and thereby PWD can be treated, ameliorated and/or prevented. As understood by any person skilled in the art, the treatment, amelioration and/or prevention of PWD can be characterized by for example increased faeces score. Preferably, in the present invention, the faeces score is increased by at least 1%, 2%, 3%, 5%, 10% or more.

In other special embodiments, the present invention provides use of the composition as described herein for improving growth performance of animals. As anticipated by any person skilled in the art, one or more of the characters of growth performance such as weight gain, daily weight gain, feed intake, daily feed intake, feed conversion ratio and/or mortality ratio of animals could be improved because of the effect of the composition as described herein in modulating growth of microorganisms in the gastrointestinal tract of animals. Preferably, any of the characters of growth performance is improved by at least 1%, 2%, 3%, 5%, 10%, 15%, 20% or more because of the composition as described herein.

In the present invention, the terms “food” and “feed” are used to refer to any component, ingredient, preparation, mixture or additive suitable for, or intended for oral intake by animals. Examples of the food and/or the feed include but are not limited to foodstuff; fodder; feed; silage; foodstuff additive; fodder additive; feed additive; silage additive; feed or food components such as crop, grain such as wet distillers grain and dried distillers grain, hay, straw, protein sources such as soy protein, starch and carbohydrates etc.; nutritional supplement; prebiotic; probiotic; intermediate/s thereof; and/or mixture/s thereof.

In the present invention, the term “animal” or “animals” refers to any animal including humans. Examples of the animal are ruminant animals such as sheep, goats, cattle, deer, yank, camel, llama and kangaroo; and non-ruminant animals such as pigs (including but not limited to piglets, growing pigs, and sows), poultry (including but not limited to turkeys, ducks, quail, guinea fowl, geese, pigeons and chicken), horses, crustaceans (such as shrimps and prawns) and fishes. Preferably, the animal is selected from the group consisting of sheep, goats, cattle, pigs (including but not limited to piglets, growing pigs, and sows), poultry (including but not limited to turkeys, ducks, quail, guinea fowl, geese, pigeons and chicken) and horses.

Preferably, in the use of the present invention, the composition modulates growth of microorganisms in a synergistic model, i.e., it provides observed value (“O”) beyond the expected value (“E”) resulting from the individual ingredient of the composition, i.e, benzoic acid and/or salt thereof and the additional organic acid and/or salt thereof, in modulating growth of microorganisms.

In the third aspect, the present invention provides a method for modulating growth of microorganisms, i.e., inhibiting the growth of pathogenic microorganisms and/or enhancing the growth of beneficial (i.e. probiotic) microorganisms, wherein the method comprises the steps of a) providing a composition as described herein; and b) contacting the composition of a) with the microorganisms.

In some embodiments, the present invention provides a method for modulating growth of microorganisms, especially for inhibiting the growth of pathogenic microorganisms, in a food and/or a feed, wherein the method comprises the steps of a) providing a composition as described herein; and b) contacting the composition of a) with the microorganisms in the food and/or the feed.

In some other embodiments, the present invention provides a method for modulating growth of microorganisms in the gastrointestinal tracts, especially in the intestinal tracts (e.g. stomach, duodenum, jejunum, illeum, cecum, and/or large intestine) of animals and/or improving gut health of animals, wherein the method comprises the steps of a) providing a composition as described herein; and b) administering to the animals the composition of a).

In some special embodiments, the present invention provides a method for the treatment, amelioration and/or prevention of post-weaning diarrhea (PWD) of animals, such as pigs, wherein the method comprises the steps of a) providing a composition as described herein; and b) administering to the animals the composition of a).

In other special embodiments, the present invention provides a method for improving growth performance of animals, wherein the method comprises the steps of a) providing a composition as described herein; and b) administering to the animals the composition of a).

Preferably, in the methods of the present invention, the composition modulates growth of microorganisms, and/or treats, ameliorates and/or prevent PWD of animals, and/or improves growth performance of animals in a synergistic model, i.e., it provides observed value (“O”) beyond the expected value (“E”) resulting from the individual ingredient of the composition, i.e, benzoic acid and/or salt thereof and the additional organic acid and/or salt thereof, in modulating growth of microorganisms, and/or treatment, amelioration and/or prevention of PWD of animals, and/or improving growth performance of animals.

In the fourth aspect, the present invention provides use of at least one additional organic acid and/or at least one salt thereof in the composition as defined above for boosting the effect of benzoic acid and/or at least one salt thereof in modulating growth of microorganisms, i.e., inhibiting the growth of pathogenic microorganisms and/or enhancing the growth of beneficial microorganisms,

Particularly, the present invention provides a method for boosting the effect of benzoic acid and/or at least one salt thereof in modulating growth of microorganisms, i.e., inhibiting the growth of pathogenic microorganisms and/or enhancing the growth of beneficial microorganisms, wherein the method comprises: a) providing a composition as described herein; and b) contacting the composition of a) with the microorganisms.

Specially, the present invention provides a method for boosting the effect of benzoic acid and/or at least one salt thereof in modulating growth of microorganisms in the gastrointestinal tracts, especially in the intestinal tracts (e.g. stomach, duodenum, jejunum, illeum, cecum, and/or large intestine) of animals, and/or in improving gut health of animals, and/or in the treatment, amelioration and/or prevention of post-weaning diarrhea (PWD) of animals, such as pigs, wherein the method comprises the steps of a) providing a composition as described herein; and b) administering to the animals the composition of a).

In the present invention, the term “boost” or “boosting” means that benzoic acid and/or at least one salt thereof and the at least one additional organic acid and/or at least one salt thereof in the composition according to the present invention modulates growth of microorganisms in a synergistic model, i.e., it provides observed value (“O”) beyond the expected value (“E”) resulting from the individual ingredient of the composition, i.e, benzoic acid and/or salt thereof and the additional organic acid and/or salt thereof, in modulating growth of microorganisms.

The present invention is further illustrated by the following examples.

### EXAMPLES

### Example 1: Modulating Growth of Microorganisms

In the example, the potential of the compositions of the present invention in modulating growth of microorganisms, i.e., inhibiting the growth of the pathogenic microorganisms and enhancing the growth of the beneficial microorganisms, was studied. In a systematic approach, growth of the pathogenic microorganisms: Escherichia coli (E. coli U5/41 (DSMZ 30083)), Salmonella enterica (S. enterica subsp. enterica serotype typhimurium SL1344 (DSMZ 24522)), Shigella sonnei (S. sonnei ATCC 29930, I virulent, WDCM 00127, CECT 4887 (DSMZ 5570)) and Clostridium perfringens (C. perfringens (CCUG 47895; type A as described in Johansson et al. 2004. Vet Microbiol. 99(3-4)): 251-257); and the beneficial microorganisms: Lactobacillus reuteri (L. reuteri F 275 (DSMZ 20016)), Enterococcus faecium (E. faecium D, serotype 11 (DSMZ 20477)) and Bacillus subtilis (B. subtilis strain Marburg (DSMZ 10)) was recorded and the synergistic effect was assessed.

In particular, the compositions of the present invention comprising either 0.2, 0.4, 0.8, 1.6, 3.1, 6.3, 12.5, 25, 50 or 100 mM of either formic acid, acetic acid, propionic acid, fumaric acid or succinic acid; and/or either 0.3, 0.6, 1.3, 2.5, 5, 10 or 20 mM of benzoic acid were prepared in Mueller-Hinton broth and sterilized by micro-filtration using a 0.22 μm filter. Either of the microorganisms listed above were cultivated in Mueller-Hinton broth (Mueller et al. 1941. Experimental Biology and Medicine 48(1): 330-333) until late exponential phase. Those liquid cultures were used to inoculate the compositions prepared as described above in 96-well microtiter plates to reach a final density of 5×10⁵ cfu/mL. As sterility control, one well containing Mueller-Hinton broth was not inoculated with microorganisms. As growth control, at least 4 wells of the 96-well microtiter plate were prepared, wherein only Mueller-Hinton broth was inoculated with either of the studied microorganisms. In other words, the growth controls were not treated with a composition as referred to herein comprising any organic acid and/or benzoic acid.

The thus inoculated microtiter plates were incubated overnight at 37 °C under anaerobic conditions, provided by a saturated nitrogen atmosphere in anaerobic jars to simulate conditions in a gastrointestinal tract. To evaluate the growth of the microorganisms, the optical density at 600 nanometers (OD600) of each well of the incubated microtiter plates was measured approximately 20-26 h after incubation start, typically after approximately 24 h, using a multimode plate reader. The initial optical density value prior to overnight incubation was subtracted from the final value obtained after the overnight incubation period to calculate the growth values. These growth values were used to calculate the amount of inhibition or enhancement compared to the growth values of the growth control wells. For instance, if the growth value of the growth control was 0.8308 after approximately 10 h, the growth value of an E. coli culture comprising 12.5 mM formic acid was 0.6624, the growth value of an E. coli culture comprising 12.5 mM benzoic acid was 0.7128, and the growth value of an E. coli culture comprising 12.5 mM formic acid and 12.5 mM benzoic acid was 0.3692, then the growth of the culture comprising 12.5 mM formic acid compared to the growth of the growth control would be calculated as 0.6624×100/0.8308=79.72%, the growth of the culture comprising 12.5 mM benzoic acid compared to the growth of the growth control would be calculated as 0.7128×100/0.8308=85.80%, and the growth of the culture comprising 12.5 mM formic acid and 12.5 mM benzoic acid compared to the growth of the growth control would be calculated as 0.3692×100/0.8308=44.44%. In other words, the composition comprising 12.5 mM formic acid inhibited the microbial growth by 100-79.92=20.28%, the composition comprising 12.5 mM benzoic acid inhibited the microbial growth by 100-85.80=14.20%, and the composition comprising 12.5 mM formic acid and 12.5 mM benzoic acid inhibited the microbial growth by 100-44.44=55.56%.

Synergistic inhibition or enhancement was assessed as published by Colby et al. (1967. Weeds 15(1): 20-22) using the following formula:

E=X+(Y/100)×(100−X)

Therein, “E” represents an expectation value; “X” represents the degree of inhibition or enhancement observed upon treatment with a substance 1 (herein: organic acid(s) and/or salt(s) thereof); and “Y” represents the degree of inhibition or enhancement observed upon treatment with a substance 2 (herein: benzoic acid and/or salt(s) thereof). The expectation value indicates the theoretical amount of inhibition or enhancement that would be observable in case of merely additive (i.e. non-synergistic) inhibition of a combination of substance 1 and substance 2. Both, “X” and “Y” are calculated in percent of inhibition or enhancement. A growth control that was not treated with substance 1 and/or substance 2, served as base value having no inhibition/enhancement, i.e. 0% of inhibition/enhancement.

In a next step, the actually observed amount of inhibition or enhancement of a combination of substance 1 and substance 2 was compared to the expectation value, and used to quantify the synergy according to the following formula:

S=O−E

Therein, “S” represents the difference in the amount of inhibition or enhancement between the calculated additive inhibition (“E”) and the actually observed amount of inhibition or enhancement (“O”); “E” represents the expectation value as described above; and “O” denotes the observed amount of inhibition or enhancement. In the event that “S” is a positive number, i.e. larger than 0, a combination of substance 1 and substance 2 was confirmed to inhibit or enhance the growth of the studied microorganisms synergistically.

For instance, in the example above, wherein a composition comprising 12.5 mM formic acid inhibited the microbial growth by 20.28%, a composition comprising 12.5 mM benzoic acid inhibited the microbial growth by 14.20%, the expectation value “E” would be calculated as 20.28+(14.20/100)×(100−20.28)=31.60%. In other words, in case of an additive or non-synergistic inhibition, a composition comprising both 12.5 mM formic acid and 12.5 mM benzoic acid would be expected to inhibit the microbial growth by 31.60%. However, in contrast to this expectation, the microbial growth was experimentally determined to be inhibited by 55.56%. Thus, “S” would be calculated as 55.56−31.60=23.96%, indicating synergistic inhibition.

Synergistic modulation of microbial growth, in particular synergistically inhibiting or enhancing the growth of the pathogenic microorganisms, was found upon treatment with compositions comprising a combination of i) one or more organic acid(s) with ii) benzoic acid. In the following Tables 1-7, examples of synergistic inhibition or enhancement of the growth of the microorganisms by compositions according to the invention are shown. Therein, the column labeled “BA (mM)” indicates the concentration of benzoic acid that was comprised in a composition as described above. The column labeled “OA (mM)” indicates the concentration of organic acid(s) that was comprised in a composition as described above. The column labeled “BA:OA” indicates the molar ratio between benzoic acid and the additional organic acid(s) that was comprised in a composition as described above. The column labeled “details” indicates which organic acid was comprised in the composition. And in addition, the synergistic inhibition effect (average “S” from two parallel experiments) is given in brackets after the organic acid with which the synergistic inhibition was observed in combination with benzoic acid.

TABLE 1
Synergistic inhibition of the growth of E. coli U5/41 (DSMZ 30083)
upon treatment with different concentrations of benzoic acid (BA)
in combination with different concentrations of organic acid.

| BA | OA | | |
|---|---|---|---|
| (mM) | (mM) | BA:OA | details |
| 20 | 100.0 | 1:5 | propionic acid (0.02) |
| 20 | 25.0 | 1:1.3 | propionic acid (0.08), fumaric acid (0.18) |
| 20 | 12.5 | 1:0.6 | acetic acid (0.02), fumaric acid (0.11), succinic acid (0.18) |
| 20 | 6.3 | 1:0.3 | formic acid (0.31), acetic acid (0.09), propionic acid (0.04), fumaric acid |
| | | | (0.04), succinic acid (0.20) |
| 20 | 3.1 | 1:0.2 | succinic acid (0.16) |
| 20 | 1.6 | 1:0.1 | acetic acid (0.02) |
| 10 | 50.0 | 1:5 | fumaric acid (16.82) |
| 10 | 25.0 | 1:2.5 | fumaric acid (40.78), succinic acid (4.46) |
| 10 | 12.5 | 1:1.3 | acetic acid (0.53), fumaric acid (32.12), succinic acid (43.46) |
| 10 | 6.3 | 1:0.6 | fumaric acid (6.27), succinic acid (51.36) |
| 10 | 3.1 | 1:0.3 | succinic acid (15.62) |
| 5.0 | 100.0 | 1:20 | formic acid (0.10), propionic acid (0.12), succinic acid (0.20) |
| 5.0 | 50.0 | 1:10 | formic acid (0.15), fumaric acid (40.23) |
| 5.0 | 25.0 | 1:5 | formic acid (0.12), fumaric acid (92.03), succinic acid (6.85) |
| 5.0 | 12.5 | 1:2.5 | formic acid (27.12), acetic acid (52.22), propionic acid (26.38), fumaric |
| | | | acid (39.82), succinic acid (81.89) |
| 5.0 | 6.3 | 1:1.3 | formic acid (53.51), acetic acid (47.12), propionic acid (2.60), fumaric |
| | | | acid (18.23), succinic acid (77.52) |
| 5.0 | 3.1 | 1:0.6 | formic acid (15.03), fumaric acid (13.33), succinic acid (16.74) |
| 5.0 | 1.6 | 1:0.3 | formic acid (11.13), fumaric acid (12.50), succinic acid (13.99) |
| 5.0 | 0.8 | 1:0.2 | formic acid (15.86), fumaric acid (14.38), succinic acid (16.25) |
| 5.0 | 0.4 | 1:0.1 | formic acid (16.34), acetic acid (0.49), fumaric acid (14.88), succinic acid |
| | | | (18.08) |
| 5.0 | 0.2 | 1:0.04 | formic acid (19.00), fumaric acid (16.73), succinic acid (23.14) |
| 2.5 | 100.0 | 1:40 | formic acid (0.15), acetic acid (0.09), propionic acid (0.24), succinic acid |
| | | | (0.01) |
| 2.5 | 50.0 | 1:20 | formic acid (0.05), propionic acid (0.03), fumaric acid (39.99) |
| 2.5 | 25.0 | 1:10 | formic acid (0.15), acetic acid (0.03), propionic acid (0.11), fumaric acid |
| | | | (55.34), succinic acid (6.78) |
| 2.5 | 12.5 | 1:5 | formic acid (27.97), acetic acid (39.29), propionic acid (33.05), fumaric |
| | | | acid (11.97), succinic acid (81.67) |
| 2.5 | 6.3 | 1:2.5 | formic acid (22.23), acetic acid (33.80), propionic acid (24.65), fumaric |
| | | | acid (15.23), succinic acid (28.00) |
| 2.5 | 3.1 | 1:1.2 | formic acid (15.18), acetic acid (26.60), propionic acid (21.18), fumaric |
| | | | acid (15.36), succinic acid (13.59) |
| 2.5 | 1.6 | 1:0.6 | formic acid (14.92), acetic acid (32.02), propionic acid (22.72), fumaric |
| | | | acid (13.00), succinic acid (19.82) |
| 2.5 | 0.8 | 1:0.3 | formic acid (15.79), acetic acid (34.23), propionic acid (25.06), fumaric |
| | | | acid (14.48), succinic acid (19.36) |
| 2.5 | 0.4 | 1:0.2 | formic acid (13.31), acetic acid (31.06), propionic acid (25.57), fumaric |
| | | | acid (13.50), succinic acid (17.96) |
| 2.5 | 0.2 | 1:0.1 | formic acid (17.26), acetic acid (29.64), propionic acid (27.01), fumaric |
| | | | acid (15.79), succinic acid (21.82) |
| 1.3 | 100.0 | 1:76.9 | formic acid (0.30), acetic acid (0.28), propionic acid (0.34), succinic acid |
| | | | (0.21) |
| 1.3 | 50.0 | 1:38.5 | formic acid (0.23), propionic acid (0.12), fumaric acid (24.44), succinic |
| | | | acid (0.05) |
| 1.3 | 25.0 | 1:19.2 | formic acid (0.24), acetic acid (0.07), propionic acid (0.18), fumaric acid |
| | | | (12.51), succinic acid (7.42) |
| 1.3 | 12.5 | 1:9.6 | formic acid (23.08), acetic acid (14.33), propionic acid (28.15), fumaric |
| | | | acid (8.63), succinic acid (34.22) |
| 1.3 | 6.3 | 1:4.8 | formic acid (23.59), acetic acid (20.24), propionic acid (24.71), fumaric |
| | | | acid (11.42), succinic acid (17.72) |
| 1.3 | 3.1 | 1:2.4 | formic acid (15.89), acetic acid (23.23), propionic acid (21.79), fumaric |
| | | | acid (10.33), succinic acid (31.64) |
| 1.3 | 1.6 | 1:1.2 | formic acid (10.38), acetic acid (25.66), propionic acid (22.61), fumaric |
| | | | acid (6.96), succinic acid (21.54) |
| 1.3 | 0.8 | 1:0.6 | formic acid (14.18), acetic acid (28.73), propionic acid (25.11), fumaric |
| | | | acid (8.59), succinic acid (21.64) |
| 1.3 | 0.4 | 1:0.3 | formic acid (12.30), acetic acid (27.77), propionic acid (28.03), fumaric |
| | | | acid (9.09), succinic acid (23.32) |
| 1.3 | 0.2 | 1:0.2 | formic acid (13.91), acetic acid (24.62), propionic acid (27.10), fumaric |
| | | | acid (10.28), succinic acid (23.37) |
| 0.6 | 100.0 | 1:166.7 | acetic acid (0.01), propionic acid (0.06), succinic acid (0.18) |
| 0.6 | 50.0 | 1:83.3 | propionic acid (0.16), fumaric acid (6.33) |
| 0.6 | 25.0 | 1:41.7 | formic acid (0.09), acetic acid (0.03), propionic acid (0.03), fumaric acid |
| | | | (3.64), succinic acid (6.90) |
| 0.6 | 12.5 | 1:20.8 | propionic acid (11.99), fumaric acid (5.17), succinic acid (16.44) |
| 0.6 | 6.3 | 1:10.5 | formic acid (17.43), acetic acid (15.10), propionic acid (22.26), fumaric |
| | | | acid (9.12), succinic acid (15.32) |
| 0.6 | 3.1 | 1:5.2 | formic acid (15.19), acetic acid (17.09), propionic acid (23.51), fumaric |
| | | | acid (9.10), succinic acid (21.06) |
| 0.6 | 1.6 | 1:2.7 | formic acid (12.74), acetic acid (19.31), propionic acid (28.85), fumaric |
| | | | acid (4.54), succinic acid (18.94) |
| 0.6 | 0.8 | 1:1.3 | formic acid (14.70), acetic acid (24.77), propionic acid (26.80), fumaric |
| | | | acid (8.31), succinic acid (21.00) |
| 0.6 | 0.4 | 1:0.7 | formic acid (15.78), acetic acid (22.05), propionic acid (29.73), fumaric |
| | | | acid 5.89( ), succinic acid (19.62) |
| 0.6 | 0.2 | 1:0.3 | formic acid (14.21), acetic acid (21.62), propionic acid (29.91), fumaric |
| | | | acid (7.92), succinic acid (20.68) |
| 0.3 | 50.0 | 1:166.7 | fumaric acid (7.51) |
| 0.3 | 25.0 | 1:83.3 | fumaric acid (9.64), succinic acid (5.80) |
| 0.3 | 12.5 | 1:41.7 | fumaric acid (12.25) |
| 0.3 | 6.3 | 1:21 | formic acid (6.35), acetic acid (9.57), propionic acid (15.95), fumaric acid |
| | | | (13.47), succinic acid (13.54) |
| 0.3 | 3.1 | 1:10.3 | formic acid (2.94), acetic acid (10.20), propionic acid (14.49), fumaric |
| | | | acid (12.00), succinic acid (12.82) |
| 0.3 | 1.6 | 1:5.3 | formic acid (2.63), acetic acid (12.18), propionic acid (15.79), fumaric |
| | | | acid (12.19), succinic acid (8.84) |
| 0.3 | 0.8 | 1:2.7 | formic acid (5.17), acetic acid (15.80), propionic acid (18.21), fumaric |
| | | | acid (14.13), succinic acid (10.07) |
| 0.3 | 0.4 | 1:1.3 | formic acid (3.70), acetic acid (15.32), propionic acid (21.39), fumaric |
| | | | acid (13.27), succinic acid (12.58) |
| 0.3 | 0.2 | 1:0.7 | formic acid (8.10), acetic acid (13.80), propionic acid (21.76), fumaric |
| | | | acid (15.60), succinic acid (12.40) |

TABLE 2
Synergistic inhibition of the growth of S. enterica subsp. enterica serotype
typhimurium SL1344 (DSMZ 24522) upon treatment with different concentrations of
benzoic acid (BA) in combination with different concentrations of organic acid.

| BA | OA | | |
|---|---|---|---|
| (mM) | (mM) | BA:OA | details |
| 20 | 100.0 | 1:5 | acetic acid (0.13), propionic acid (0.16) |
| 20 | 25.0 | 1:1.3 | propionic acid (0.05) |
| 20 | 12.5 | 1:0.6 | propionic acid (0.10) |
| 20 | 6.3 | 1:0.3 | acetic acid (0.26), propionic acid (0.39) |
| 20 | 3.1 | 1:0.2 | acetic acid (0.32), propionic acid (0.41) |
| 20 | 1.6 | 1:0.1 | acetic acid (0.39), propionic acid (0.38) |
| 20 | 0.8 | 1:0.04 | acetic acid (0.23), propionic acid (0.35) |
| 20 | 0.4 | 1:0.02 | propionic acid (0.25) |
| 10 | 50.0 | 1:5 | propionic acid (0.03), fumaric acid (2.26), succinic acid (0.11) |
| 10 | 25.0 | 1:2.5 | propionic acid (0.10), fumaric acid (10.06), succinic acid (1.13) |
| 10 | 12.5 | 1:1.3 | acetic acid (0.06), propionic acid (0.21), fumaric acid (11.18), succinic |
| | | | acid (5.27) |
| 10 | 6.3 | 1:0.6 | formic acid (0.11), acetic acid (0.45), propionic acid (0.40), fumaric acid |
| | | | (11.14), succinic acid (13.68) |
| 10 | 3.1 | 1:0.3 | succinic acid (13.79) |
| 5.0 | 100.0 | 1:20 | succinic acid (0.14) |
| 5.0 | 50.0 | 1:10 | propionic acid (0.01), fumaric acid (19.65), succinic acid (0.40) |
| 5.0 | 25.0 | 1:5 | formic acid (0.17), fumaric acid (83.85), succinic acid (6.03) |
| 5.0 | 12.5 | 1:2.5 | acetic acid (5.06), propionic acid (4.84), fumaric acid (78.48), succinic |
| | | | acid (31.23) |
| 5.0 | 6.3 | 1:1.3 | formic acid (55.97), fumaric acid (38.44), succinic acid (91.86) |
| 5.0 | 3.1 | 1:0.6 | formic acid (1.78), fumaric acid (19.68), succinic acid (33.40) |
| 5.0 | 1.6 | 1:0.3 | fumaric acid (11.25), succinic acid (12.79) |
| 5.0 | 0.8 | 1:0.2 | formic acid (0.88), fumaric acid (7.93), succinic acid (12.38) |
| 5.0 | 0.4 | 1:0.1 | formic acid (0.34), fumaric acid (6.20), succinic acid (6.55) |
| 5.0 | 0.2 | 1:0.04 | formic acid (3.34), fumaric acid (8.68), succinic acid (8.06) |
| 2.5 | 100.0 | 1:40 | succinic acid (0.18) |
| 2.5 | 50.0 | 1:20 | propionic acid (0.11), fumaric acid (22.77), succinic acid (0.35) |
| 2.5 | 25.0 | 1:10 | formic acid (0.38), fumaric acid (84.70), succinic acid (6.71) |
| 2.5 | 12.5 | 1:5 | acetic acid (20.97), propionic acid (11.77), fumaric acid (35.69), succinic |
| | | | acid (35.04) |
| 2.5 | 6.3 | 1:2.5 | formic acid (14.72), acetic acid (2.61), fumaric acid (23.01), succinic acid |
| | | | (46.50) |
| 2.5 | 3.1 | 1:1.2 | formic acid (10.75), fumaric acid (17.20), succinic acid (21.91) |
| 2.5 | 1.6 | 1:0.6 | formic acid (8.31), fumaric acid (11.53), succinic acid (20.76) |
| 2.5 | 0.8 | 1:0.3 | formic acid (19.74), fumaric acid (12.15), succinic acid (15.31) |
| 2.5 | 0.4 | 1:0.2 | formic acid (20.64), fumaric acid (9.59), succinic acid (13.63) |
| 2.5 | 0.2 | 1:0.1 | formic acid (23.59), fumaric acid (18.07), succinic acid (14.57) |
| 1.3 | 100.0 | 1:76.9 | propionic acid (0.02), succinic acid (0.18) |
| 1.3 | 50.0 | 1:38.5 | formic acid (0.14), propionic acid (0.12), fumaric acid (21.64), succinic |
| | | | acid (0.38) |
| 1.3 | 25.0 | 1:19.2 | formic acid (0.44), fumaric acid (39.88), succinic acid (6.75) |
| 1.3 | 12.5 | 1:9.6 | acetic acid (11.17), fumaric acid (14.58), succinic acid (22.93) |
| 1.3 | 6.3 | 1:4.8 | formic acid (11.87), acetic acid (11.32), propionic acid (11.96), fumaric |
| | | | acid (10.53), succinic acid (22.56) |
| 1.3 | 3.1 | 1:2.4 | formic acid (14.46), acetic acid (5.21), propionic acid (9.77), fumaric acid |
| | | | (10.27), succinic acid (15.56) |
| 1.3 | 1.6 | 1:1.2 | formic acid (17.42), acetic acid (10.31), propionic acid (11.94), fumaric |
| | | | acid (6.76), succinic acid (16.57) |
| 1.3 | 0.8 | 1:0.6 | formic acid (25.01), acetic acid (10.50), propionic acid (12.70), fumaric |
| | | | acid (6.76), succinic acid (15.61) |
| 1.3 | 0.4 | 1:0.3 | formic acid (26.62), acetic acid (6.76), propionic acid (12.92), fumaric |
| | | | acid (5.04), succinic acid (14.40) |
| 1.3 | 0.2 | 1:0.2 | formic acid (30.85), acetic acid (10.16), propionic acid (19.61), fumaric |
| | | | acid (10.04), succinic acid (15.63) |
| 0.6 | 100.0 | 1:166.7 | succinic acid (0.27) |
| 0.6 | 50.0 | 1:83.3 | formic acid (0.13), acetic acid (0.01), propionic acid (0.21), fumaric acid |
| | | | (15.43), succinic acid (0.28) |
| 0.6 | 25.0 | 1:41.7 | formic acid (0.42), acetic acid 0.05( ), propionic acid (0.07), fumaric acid |
| | | | (18.05), succinic acid (6.83) |
| 0.6 | 12.5 | 1:20.8 | acetic acid (1.12), fumaric acid (12.00), succinic acid (12.70) |
| 0.6 | 6.3 | 1:10.5 | formic acid (8.94), acetic acid (8.62), propionic acid (19.82), fumaric acid |
| | | | (12.65), succinic acid (15.12) |
| 0.6 | 3.1 | 1:5.2 | formic acid (9.95), acetic acid (8.67), propionic acid (20.24), fumaric acid |
| | | | (13.28), succinic acid (15.31) |
| 0.6 | 1.6 | 1:2.7 | formic acid (12.13), acetic acid (9.10), propionic acid (25.00), fumaric |
| | | | acid (11.82), succinic acid (16.68) |
| 0.6 | 0.8 | 1:1.3 | formic acid (21.42), acetic acid (12.43), propionic acid (23.64), fumaric |
| | | | acid (10.76), succinic acid (18.21) |
| 0.6 | 0.4 | 1:0.7 | formic acid (21.67), acetic acid (9.05), propionic acid (28.23), fumaric |
| | | | acid (11.26), succinic acid (18.65) |
| 0.6 | 0.2 | 1:0.3 | formic acid (23.67), acetic acid (11.02), propionic acid (26.18), fumaric |
| | | | acid (14.92), succinic acid (19.97) |
| 0.3 | 100.0 | 1:333.3 | fumaric acid (0.01), succinic acid (0.14) |
| 0.3 | 50.0 | 1:166.7 | formic acid (0.24), acetic acid (0.03), fumaric acid (4.42), succinic acid |
| | | | (0.12) |
| 0.3 | 25.0 | 1:83.3 | formic acid (0.40), acetic acid (0.12), fumaric acid (16.86), succinic acid |
| | | | (7.13) |
| 0.3 | 12.5 | 1:41.7 | fumaric acid (15.22), succinic acid (5.22) |
| 0.3 | 6.3 | 1:21 | formic acid (11.46), acetic acid (10.14), propionic acid (19.79), fumaric |
| | | | acid (16.76), succinic acid (14.28) |
| 0.3 | 3.1 | 1:10.3 | formic acid (11.25), acetic acid (14.87), propionic acid (25.15), fumaric |
| | | | acid (19.08), succinic acid (12.76) |
| 0.3 | 1.6 | 1:5.3 | formic acid (15.83), acetic acid (16.49), propionic acid (16.17), fumaric |
| | | | acid (16.48), succinic acid (14.93) |
| 0.3 | 0.8 | 1:2.7 | formic acid (24.50), acetic acid 18.00( ), propionic acid (27.86), fumaric |
| | | | acid (17.28), succinic acid (19.13) |
| 0.3 | 0.4 | 1:1.3 | formic acid (22.50), acetic acid (9.53), propionic acid (29.57), fumaric |
| | | | acid (16.21), succinic acid (16.88) |
| 0.3 | 0.2 | 1:0.7 | formic acid (24.86), acetic acid (13.81), propionic acid (29.58), fumaric |
| | | | acid (20.26), succinic acid (17.29) |

TABLE 3
Synergistic inhibition of the growth of S. sonnei ATCC 29930, I virulent, WDCM
00127, CECT 4887 (DSMZ 5570) upon treatment with different concentrations of benzoic
acid (BA) in combination with different concentrations of organic acid.

| BA | OA | | |
|---|---|---|---|
| (mM) | (mM) | BA:OA | details |
| 20 | 100.0 | 1:5 | formic acid (0.070), acetic acid (2.12), propionic acid (2.03), succinic acid |
| | | | (1.62) |
| 20 | 50.0 | 1:2.5 | formic acid (1.27), acetic acid (2.20), propionic acid (2.03), succinic acid |
| | | | (1.88) |
| 20 | 25.0 | 1:1.3 | formic acid (1.39), acetic acid (2.12), propionic acid (1.90), fumaric acid |
| | | | (0.12), succinic acid (1.91) |
| 20 | 12.5 | 1:0.6 | formic acid (1.27), acetic acid (1.51), propionic acid (1.55), fumaric acid |
| | | | (0.22), succinic acid (1.13) |
| 20 | 6.3 | 1:0.3 | acetic acid (0.25), propionic acid (0.48) |
| 20 | 3.1 | 1:0.2 | acetic acid (0.42), propionic acid (0.34), fumaric acid (0.06) |
| 20 | 1.6 | 1:0.1 | formic acid (0.17), acetic acid (0.54), propionic acid (0.56), fumaric acid |
| | | | (0.22), succinic acid (0.19) |
| 20 | 0.8 | 1:0.04 | formic acid (0.18), acetic acid (0.71), propionic acid (0.67), fumaric acid |
| | | | (0.39), succinic acid (0.58) |
| 20 | 0.4 | 1:0.02 | formic acid (0.11), acetic acid (0.74), propionic acid (0.45), fumaric acid |
| | | | (0.34), succinic acid (0.57) |
| 20 | 0.2 | 1:0.01 | formic acid (0.25), acetic acid (0.62), propionic acid (0.55), fumaric acid |
| | | | (0.42), succinic acid (0.35) |
| 10 | 100.0 | 1:10 | formic acid (0.65), acetic acid (1.08), propionic acid (0.71), succinic acid |
| | | | (0.89) |
| 10 | 50.0 | 1:5 | formic acid (1.00), acetic acid (0.92), propionic acid (0.71), succinic acid |
| | | | (1.02) |
| 10 | 25.0 | 1:2.5 | formic acid (0.58), acetic acid (0.75), propionic acid (0.57), fumaric acid |
| | | | (0.36), succinic acid (0.76) |
| 10 | 12.5 | 1:1.3 | formic acid (0.69), acetic acid (1.14), propionic acid (0.96), fumaric acid |
| | | | (1.17), succinic acid (1.56) |
| 10 | 6.3 | 1:0.6 | formic acid (0.27), acetic acid (2.68), propionic acid (0.76), fumaric acid |
| | | | (0.94), succinic acid (1.34) |
| 10 | 3.1 | 1:0.3 | formic acid (0.66), acetic acid (2.70), propionic acid (1.09), fumaric acid |
| | | | (0.64), succinic acid (2.28) |
| 10 | 1.6 | 1:0.2 | formic acid (0.57), acetic acid (2.28), propionic acid (0.98), fumaric acid |
| | | | (0.34), succinic acid (1.58) |
| 10 | 0.8 | 1:0.1 | formic acid (0.28), acetic acid (2.33), propionic acid (0.67), fumaric acid |
| | | | (0.41), succinic acid (0.43) |
| 10 | 0.4 | 1:0 | acetic acid (1.46), propionic acid (0.48) |
| 5.0 | 100.0 | 1:20 | formic acid (0.56), acetic acid (1.01), propionic acid (0.69), succinic acid |
| | | | (1.11) |
| 5.0 | 50.0 | 1:10 | formic acid (0.97), acetic acid (0.58), propionic acid (0.46), fumaric acid |
| | | | (0.07), succinic acid (0.84) |
| 5.0 | 25.0 | 1:5 | formic acid (0.34), acetic acid (0.52), propionic acid (0.24), fumaric acid |
| | | | (46.25), succinic acid (0.30) |
| 5.0 | 12.5 | 1:2.5 | formic acid (0.11), acetic acid (8.70), propionic acid (8.80), fumaric acid |
| | | | (56.78), succinic acid (26.06) |
| 5.0 | 6.3 | 1:1.3 | formic acid (30.18), acetic acid (28.42), propionic acid (36.21), fumaric |
| | | | acid (31.39), succinic acid (61.39) |
| 5.0 | 3.1 | 1:0.6 | formic acid (10.65), acetic acid (7.72), propionic acid (19.71), fumaric |
| | | | acid (16.93), succinic acid (43.75) |
| 5.0 | 1.6 | 1:0.3 | propionic acid (5.22), fumaric acid (15.04), succinic acid (25.38) |
| 5.0 | 0.8 | 1:0.2 | fumaric acid (4.81), succinic acid (20.47) |
| 5.0 | 0.4 | 1:0.1 | fumaric acid (12.61), succinic acid (30.62) |
| 5.0 | 0.2 | 1:0 | fumaric acid (5.30), succinic acid (20.02) |
| 2.5 | 100.0 | 1:40 | formic acid (1.62), acetic acid (0.86), propionic acid (0.62), succinic acid |
| | | | (1.12) |
| 2.5 | 50.0 | 1:20 | formic acid (0.82), acetic acid (0.68), propionic acid (0.41), fumaric acid |
| | | | (0.23), succinic acid (0.66) |
| 2.5 | 25.0 | 1:10 | formic acid (0.56), acetic acid (0.42), propionic acid (0.25), fumaric acid |
| | | | (80.98), succinic acid (0.12) |
| 2.5 | 12.5 | 1:5 | acetic acid (16.35), propionic acid (12.14), fumaric acid (67.93), succinic |
| | | | acid (34.87) |
| 2.5 | 6.3 | 1:2.5 | formic acid (54.69), acetic acid (21.34), propionic acid (18.30), fumaric |
| | | | acid (37.10), succinic acid (65.67) |
| 2.5 | 3.1 | 1:1.2 | formic acid (4.75), acetic acid (0.60), fumaric acid (27.55), succinic acid |
| | | | (39.02) |
| 2.5 | 1.6 | 1:0.6 | formic acid (5.98), propionic acid (6.90), fumaric acid (31.84), succinic |
| | | | acid (25.03) |
| 2.5 | 0.8 | 1:0.3 | acetic acid (3.70), fumaric acid (11.82), succinic acid (24.60) |
| 2.5 | 0.4 | 1:0.2 | acetic acid (0.77), fumaric acid (23.00), succinic acid (28.47) |
| 2.5 | 0.2 | 1:0.1 | fumaric acid (12.83), succinic acid (9.05) |
| 1.3 | 100.0 | 1:76.9 | formic acid (1.56), acetic acid (0.78), propionic acid (0.55), succinic acid |
| | | | (1.23) |
| 1.3 | 50.0 | 1:38.5 | formic acid (0.92), acetic acid (0.66), propionic acid (0.57), fumaric acid |
| | | | (0.38), succinic acid (0.78) |
| 1.3 | 25.0 | 1:19.2 | formic acid (0.55), acetic acid (0.33), fumaric acid (65.89), succinic acid |
| | | | (0.19) |
| 1.3 | 12.5 | 1:9.6 | acetic acid (18.46), propionic acid (11.93), fumaric acid (24.21), succinic |
| | | | acid (40.30) |
| 1.3 | 6.3 | 1:4.8 | formic acid (39.05), acetic acid (13.08), propionic acid (3.88), fumaric |
| | | | acid (6.88), succinic acid (45.19) |
| 1.3 | 3.1 | 1:2.4 | formic acid (4.38), acetic acid (0.72), propionic acid (1.92), succinic acid |
| | | | (40.69) |
| 1.3 | 1.6 | 1:1.2 | fumaric acid (7.02), succinic acid (17.34) |
| 1.3 | 0.8 | 1:0.6 | formic acid (4.37) |
| 1.3 | 0.4 | 1:0.3 | succinic acid (39.54) |
| 1.3 | 0.2 | 1:0.2 | succinic acid (11.78) |
| 0.6 | 100.0 | 1:166.7 | formic acid (1.48), acetic acid (1.02), propionic acid (0.57), succinic acid |
| | | | (1.03) |
| 0.6 | 50.0 | 1:83.3 | formic acid (0.88), acetic acid (0.69), propionic acid (0.48), fumaric acid |
| | | | (0.56), succinic acid (0.80) |
| 0.6 | 25.0 | 1:41.7 | formic acid (0.53), acetic acid (0.39), propionic acid (0.62), fumaric acid |
| | | | (42.62), succinic acid (0.03) |
| 0.6 | 12.5 | 1:20.8 | acetic acid (16.65), propionic acid (0.28), fumaric acid (37.16), succinic |
| | | | acid (34.71) |
| 0.6 | 6.3 | 1:10.5 | formic acid (36.13), acetic acid (24.78), propionic acid (0.27), succinic |
| | | | acid (21.34) |
| 0.6 | 3.1 | 1:5.2 | formic acid (2.04), acetic acid (8.35), succinic acid (26.95) |
| 0.6 | 1.6 | 1:2.7 | formic acid (3.93), propionic acid (5.30), succinic acid (30.77) |
| 0.6 | 0.8 | 1:1.3 | formic acid (1.14), propionic acid (1.81), succinic acid (8.20) |
| 0.6 | 0.4 | 1:0.7 | acetic acid (14.19), succinic acid (51.80) |
| 0.6 | 0.2 | 1:0.3 | acetic acid (10.96), succinic acid (28.22) |
| 0.3 | 100.0 | 1:333.3 | formic acid (0.69), acetic acid (0.34), succinic acid (0.74) |
| 0.3 | 50.0 | 1:166.7 | formic acid (0.02), acetic acid (0.19) |
| 0.3 | 25.0 | 1:83.3 | formic acid (0.20), acetic acid (0.13), fumaric acid (32.98) |
| 0.3 | 12.5 | 1:41.7 | acetic acid (14.19), propionic acid (3.30), fumaric acid (35.37), succinic |
| | | | acid (22.83) |
| 0.3 | 6.3 | 1:21 | formic acid (19.83), acetic acid (33.47), fumaric acid (22.22), succinic |
| | | | acid (35.33) |
| 0.3 | 3.1 | 1:10.3 | propionic acid (2.93), fumaric acid (1.41), succinic acid (49.68) |
| 0.3 | 1.6 | 1:5.3 | formic acid (2.26), propionic acid (23.81), fumaric acid (15.38), succinic |
| | | | acid (34.40) |
| 0.3 | 0.8 | 1:2.7 | formic acid (12.42), propionic acid (23.43), fumaric acid (2.97), succinic |
| | | | acid (30.84) |
| 0.3 | 0.4 | 1:1.3 | fumaric acid (29.30), succinic acid (49.90) |
| 0.3 | 0.2 | 1:0.7 | fumaric acid (21.52), succinic acid (43.03) |

TABLE 4
Synergistic inhibition of the growth of C. perfringens (CCUG 47895; type A as described in
Johansson et al. 2004. Vet Microbiol. 99(3-4): 251-257) upon treatment with different concentrations
of benzoic acid (BA) in combination with different concentrations of organic acid.

| BA | OA | | |
|---|---|---|---|
| (mM) | (mM) | BA:OA | details |
| 20 | 50.0 | 1:2.5 | fumaric acid (1.49) |
| 20 | 25.0 | 1:1.3 | acetic acid (2.59), propionic acid (9.07), fumaric acid (3.03) |
| 20 | 12.5 | 1:0.6 | formic acid (4.64), acetic acid (4.15), propionic acid (8.00), fumaric acid |
| | | | (2.80), succinic acid (2.02) |
| 20 | 6.3 | 1:0.3 | formic acid (3.08), acetic acid (2.26), propionic acid (4.39), fumaric acid |
| | | | (0.63), succinic acid (3.34) |
| 20 | 3.1 | 1:0.2 | acetic acid (0.26), propionic acid (2.27) |
| 20 | 1.6 | 1:0.1 | formic acid (1.63), acetic acid (1.85), propionic acid (3.79), succinic acid |
| | | | (0.90) |
| 20 | 0.8 | 1:0.04 | formic acid (1.45), acetic acid (2.13), propionic acid (4.75), fumaric acid |
| | | | (0.26), succinic acid (2.35) |
| 20 | 0.4 | 1:0.02 | formic acid (2.17), acetic acid (1.55), propionic acid (5.04), fumaric acid |
| | | | (1.14), succinic acid (3.78) |
| 20 | 0.2 | 1:0.01 | formic acid (1.01), acetic acid (3.32), propionic acid (4.06), fumaric acid |
| | | | (0.82), succinic acid (3.04) |
| 10 | 50.0 | 1:5 | fumaric acid (25.30) |
| 10 | 25.0 | 1:2.5 | formic acid (5.96), acetic acid (18.59), propionic acid (41.96), fumaric |
| | | | acid (6.02), succinic acid (11.39) |
| 10 | 12.5 | 1:1.3 | formic acid (15.62), acetic acid (23.41), propionic acid (18.32), succinic |
| | | | acid (31.30) |
| 10 | 6.3 | 1:0.6 | propionic acid (0.73), succinic acid (10.56) |
| 10 | 3.1 | 1:0.3 | propionic acid (3.88), succinic acid (5.32) |
| 10 | 1.6 | 1:0.2 | formic acid (2.00), propionic acid (7.83) |
| 10 | 0.8 | 1:0.1 | formic acid (4.10), acetic acid (1.52), propionic acid (11.98) |
| 10 | 0.4 | 1:0.04 | formic acid (4.39), acetic acid (6.29), propionic acid (11.09) |
| 10 | 0.2 | 1:0.02 | formic acid (7.62), acetic acid (12.64), propionic acid (12.63), fumaric |
| | | | acid (1.41) |
| 5.0 | 50.0 | 1:10 | fumaric acid (1.83) |
| 5.0 | 25.0 | 1:5 | formic acid (10.15), acetic acid (4.11), propionic acid (16.87), fumaric |
| | | | acid (1.21), succinic acid (15.69) |
| 5.0 | 12.5 | 1:2.5 | formic acid (14.82), acetic acid (22.36), propionic acid (9.96), fumaric |
| | | | acid (14.99), succinic acid (13.12) |
| 5.0 | 6.3 | 1:1.3 | formic acid (14.03), acetic acid (12.19), succinic acid (7.77) |
| 5.0 | 3.1 | 1:0.6 | succinic acid (9.91) |
| 5.0 | 0.4 | 1:0.1 | fumaric acid (10.98) |
| 5.0 | 0.2 | 1:0.04 | fumaric acid (1.55) |
| 2.5 | 100.0 | 1:40 | succinic acid (0.21) |
| 2.5 | 50.0 | 1:20 | fumaric acid (13.17) |
| 2.5 | 25.0 | 1:10 | formic acid (9.13), acetic acid (2.51), propionic acid (16.64), fumaric acid |
| | | | (14.94), succinic acid (15.34) |
| 2.5 | 12.5 | 1:5 | formic acid (30.62), acetic acid (20.19), propionic acid (12.87), fumaric |
| | | | acid (12.77), succinic acid (32.17) |
| 2.5 | 6.3 | 1:2.5 | formic acid (16.30), acetic acid (16.01), succinic acid (35.09) |
| 2.5 | 3.1 | 1:1.2 | formic acid (4.22), fumaric acid (10.40), succinic acid (35.49) |
| 2.5 | 1.6 | 1:0.6 | fumaric acid (10.74), succinic acid (28.17) |
| 2.5 | 0.8 | 1:0.3 | succinic acid (27.45) |
| 2.5 | 0.4 | 1:0.2 | formic acid (1.32), fumaric acid (25.50), succinic acid (38.70) |
| 2.5 | 0.2 | 1:0.1 | fumaric acid (15.91), succinic acid (18.66) |
| 1.3 | 100.0 | 1:76.9 | succinic acid (1.92) |
| 1.3 | 50.0 | 1:38.5 | fumaric acid (11.10) |
| 1.3 | 25.0 | 1:19.2 | formic acid (5.63), acetic acid (5.25), propionic acid (33.28), succinic acid |
| | | | (7.25) |
| 1.3 | 12.5 | 1:9.6 | formic acid (30.64), acetic acid (3.09), propionic acid (24.56), succinic |
| | | | acid (28.12) |
| 1.3 | 6.3 | 1:4.8 | formic acid (3.50), succinic acid (7.93) |
| 1.3 | 3.1 | 1:2.4 | succinic acid (2.01) |
| 1.3 | 0.4 | 1:0.3 | formic acid (5.22), fumaric acid (10.77), succinic acid (14.08) |
| 1.3 | 0.2 | 1:0.2 | succinic acid (13.17) |
| 0.6 | 100.0 | 1:166.7 | succinic acid (1.79) |
| 0.6 | 50.0 | 1:83.3 | fumaric acid (0.05) |
| 0.6 | 25.0 | 1:41.7 | formic acid (3.19), acetic acid (7.47), propionic acid (42.12), succinic acid |
| | | | (9.92) |
| 0.6 | 12.5 | 1:20.8 | formic acid (23.94), acetic acid (17.14), propionic acid (32.25), succinic |
| | | | acid (16.26) |
| 0.6 | 6.3 | 1:10.5 | formic acid (2.90), propionic acid (0.30), fumaric acid (13.57) |
| 0.6 | 3.1 | 1:5.2 | formic acid (13.52), propionic acid (5.60), fumaric acid (5.45), succinic |
| | | | acid (10.60) |
| 0.6 | 1.6 | 1:2.7 | formic acid (3.91), propionic acid (10.67), fumaric acid (9.25) |
| 0.6 | 0.8 | 1:1.3 | formic acid (1.98), propionic acid (11.09), succinic acid (9.77) |
| 0.6 | 0.4 | 1:0.7 | propionic acid (23.34), fumaric acid (15.84), succinic acid (40.36) |
| 0.6 | 0.2 | 1:0.3 | propionic acid (2.57), fumaric acid (16.22), succinic acid (3.05) |
| 0.3 | 100.0 | 1:333.3 | succinic acid (1.79) |
| 0.3 | 50.0 | 1:166.7 | fumaric acid (0.93) |
| 0.3 | 25.0 | 1:83.3 | formic acid (5.36), acetic acid (2.47), propionic acid (21.63), fumaric acid |
| | | | (0.80), succinic acid (9.73) |
| 0.3 | 12.5 | 1:41.7 | formic acid (18.88), acetic acid (20.17), propionic acid (12.61), succinic |
| | | | acid (12.14) |
| 0.3 | 6.3 | 1:21 | formic acid (7.66), acetic acid (13.24), succinic acid (9.63) |
| 0.3 | 3.1 | 1:10.3 | succinic acid (19.82) |
| 0.3 | 1.6 | 1:5.3 | fumaric acid (3.73) |
| 0.3 | 0.8 | 1:2.7 | acetic acid (17.41), succinic acid (33.73) |
| 0.3 | 0.4 | 1:1.3 | formic acid (0.73), propionic acid (10.79), fumaric acid (12.44), succinic |
| | | | acid (40.08) |
| 0.3 | 0.2 | 1:0.7 | formic acid (17.46), acetic acid (4.89), fumaric acid (16.78), succinic acid |
| | | | (13.57) |

TABLE 5
Synergistic enhancement of the growth of L. reuteri F 275 (DSMZ
20016) upon treatment with different concentrations of benzoic acid
(BA) in combination with different concentrations of organic acid.

| BA | OA | | |
|---|---|---|---|
| (mM) | (mM) | BA:OA | details |
| 20 | 100.0 | 1:5 | formic acid (0.14), acetic acid (12.17), propionic acid (0.39), |
| | | | fumaric acid (21.70) |
| 20 | 50.0 | 1:2.5 | formic acid (0.52), acetic acid (16.12), propionic acid (13.93), |
| | | | fumaric acid (134.32), succinic acid (5.30) |
| 20 | 25.0 | 1:1.3 | formic acid (43.03), acetic acid (15.52), propionic acid (13.25), |
| | | | fumaric acid (120.94), succinic acid (127.67) |
| 20 | 12.5 | 1:0.6 | formic acid (51.77), acetic acid (13.14), propionic acid (6.88), |
| | | | fumaric acid (92.37), succinic acid (129.20) |
| 20 | 6.3 | 1:0.3 | formic acid (48.20), acetic acid (6.51), fumaric acid (33.49), |
| | | | succinic acid (104.14) |
| 20 | 3.1 | 1:0.2 | formic acid (31.35), fumaric acid (9.24), succinic acid (42.34) |
| 20 | 1.6 | 1:0.1 | succinic acid (0.74) |
| 10 | 100.0 | 1:10 | acetic acid (115.24), fumaric acid (22.51) |
| 10 | 50.0 | 1:5 | formic acid (0.69), acetic acid (165.38), propionic acid (141.35), |
| | | | fumaric acid (133.39), succinic acid (6.22) |
| 10 | 25.0 | 1:2.5 | formic acid (113.75), acetic acid (114.79), propionic acid (46.33), |
| | | | fumaric acid (22.22), succinic acid (154.89) |
| 10 | 12.5 | 1:1.3 | formic acid (60.01), acetic acid (49.95), propionic acid (8.40), |
| | | | succinic acid (62.98) |
| 10 | 6.3 | 1:0.6 | formic acid (14.96), acetic acid (17.27), succinic acid (23.24) |
| 10 | 3.1 | 1:0.3 | formic acid (7.54), acetic acid (9.73), succinic acid (7.92) |
| 10 | 1.6 | 1:0.2 | formic acid (5.58), acetic acid (7.25), succinic acid (3.41) |
| 10 | 0.8 | 1:0.1 | formic acid (5.74), acetic acid (4.84), succinic acid (3.14) |
| 10 | 0.4 | 1:0.04 | formic acid (14.95), acetic acid (1.05), succinic acid (2.74) |
| 10 | 0.2 | 1:0.02 | formic acid (6.66), succinic acid (0.70) |
| 5.0 | 100.0 | 1:20 | acetic acid (104.28), fumaric acid (20.10), succinic acid (0.11) |
| 5.0 | 50.0 | 1:10 | acetic acid (73.76), propionic acid (49.88), fumaric acid (6.68), |
| | | | succinic acid (5.85) |
| 5.0 | 25.0 | 1:5 | formic acid (102.13), acetic acid (28.23), propionic acid (7.38), |
| | | | succinic acid (37.65) |
| 5.0 | 12.5 | 1:2.5 | formic acid (3.30), acetic acid (9.96), succinic acid (17.29) |
| 5.0 | 6.3 | 1:1.3 | formic acid (4.82) |
| 5.0 | 3.1 | 1:0.6 | formic acid (4.60) |
| 5.0 | 1.6 | 1:0.3 | formic acid (5.28) |
| 5.0 | 0.8 | 1:0.2 | formic acid (10.26), acetic acid (2.24) |
| 5.0 | 0.4 | 1:0.1 | formic acid (10.53), acetic acid (0.97) |
| 5.0 | 0.2 | 1:0 | formic acid (7.24), propionic acid (0.98) |
| 2.5 | 100.0 | 1:40 | acetic acid (96.57), propionic acid (21.88), succinic acid (0.38) |
| 2.5 | 50.0 | 1:20 | acetic acid (18.39), propionic acid (1.75), succinic acid (5.72) |
| 2.5 | 25.0 | 1:10 | formic acid (16.92), acetic acid (8.09), propionic acid (0.36), |
| | | | succinic acid (12.71) |
| 2.5 | 12.5 | 1:5 | formic acid (3.64), acetic acid (6.53), succinic acid (12.34) |
| 2.5 | 6.3 | 1:2.5 | formic acid (6.93), succinic acid (3.77) |
| 2.5 | 3.1 | 1:1.2 | formic acid (1.33), propionic acid (0.42), succinic acid (1.71) |
| 2.5 | 1.6 | 1:0.6 | formic acid (2.61), succinic acid (1.83) |
| 2.5 | 0.8 | 1:0.3 | formic acid (8.83), acetic acid (2.95), succinic acid (2.56) |
| 2.5 | 0.4 | 1:0.2 | formic acid (10.78), acetic acid (1.89), succinic acid (1.58) |
| 2.5 | 0.2 | 1:0.1 | formic acid (8.46), propionic acid (3.20), succinic acid (1.35) |
| 1.3 | 100.0 | 1:76.9 | acetic acid (55.74), propionic acid (0.48), fumaric acid (21.32), |
| | | | succinic acid (0.28) |
| 1.3 | 50.0 | 1:38.5 | acetic acid (12.79), succinic acid (5.76) |
| 1.3 | 25.0 | 1:19.2 | formic acid (3.63), acetic acid (8.79), succinic acid (11.91) |
| 1.3 | 12.5 | 1:9.6 | formic acid (2.30), acetic acid (9.19), succinic acid (10.09) |
| 1.3 | 6.3 | 1:4.8 | formic acid (0.91), acetic acid (6.28), succinic acid (2.10) |
| 1.3 | 3.1 | 1:2.4 | acetic acid (0.68), succinic acid (3.41) |
| 1.3 | 1.6 | 1:1.2 | acetic acid (4.35), succinic acid (5.65) |
| 1.3 | 0.8 | 1:0.6 | formic acid (2.03), acetic acid (0.62), succinic acid (0.93) |
| 1.3 | 0.4 | 1:0.3 | formic acid (6.53), acetic acid (3.81), succinic acid (2.99) |
| 1.3 | 0.2 | 1:0.2 | formic acid (5.32), succinic acid (1.03) |
| 0.6 | 100.0 | 1:166.7 | acetic acid (90.21), propionic acid (0.17), fumaric acid (13.39), |
| | | | succinic acid (0.68) |
| 0.6 | 50.0 | 1:83.3 | acetic acid (14.64), succinic acid (4.00) |
| 0.6 | 25.0 | 1:41.7 | acetic acid (9.20), succinic acid (9.48) |
| 0.6 | 12.5 | 1:20.8 | formic acid (1.45), acetic acid (9.82), succinic acid (9.72) |
| 0.6 | 6.3 | 1:10.5 | formic acid (4.04), acetic acid (8.15), succinic acid (5.43) |
| 0.6 | 3.1 | 1:5.2 | formic acid (0.19), succinic acid (19.05) |
| 0.6 | 1.6 | 1:2.7 | formic acid (1.57), acetic acid (6.33), succinic acid (4.25) |
| 0.6 | 0.8 | 1:1.3 | formic acid (1.45), acetic acid (5.71), succinic acid (4.01) |
| 0.6 | 0.4 | 1:0.7 | formic acid (8.01), acetic acid (8.14), succinic acid (3.83) |
| 0.6 | 0.2 | 1:0.3 | formic acid (9.00), acetic acid (2.99), succinic acid (4.22) |
| 0.3 | 100.0 | 1:333.3 | formic acid (0.28), acetic acid (25.82), fumaric acid (21.69), |
| | | | succinic acid 0.58( ) |
| 0.3 | 50.0 | 1:166.7 | formic acid (1.23), acetic acid (9.94), succinic acid (6.47) |
| 0.3 | 25.0 | 1:83.3 | formic acid (1.77), acetic acid (12.07), succinic acid (5.77) |
| 0.3 | 12.5 | 1:41.7 | formic acid (4.31), acetic acid (11.95), succinic acid (4.17) |
| 0.3 | 6.3 | 1:21 | formic acid (4.90), acetic acid (11.51), succinic acid (1.70) |
| 0.3 | 3.1 | 1:10.3 | formic acid (3.52), acetic acid (8.53), succinic acid (0.10) |
| 0.3 | 1.6 | 1:5.3 | formic acid (0.40), acetic acid (9.57), succinic acid (0.54) |
| 0.3 | 0.8 | 1:2.7 | formic acid 8.26( ), acetic acid (7.99) |
| 0.3 | 0.4 | 1:1.3 | formic acid (9.15), acetic acid (8.85) |
| 0.3 | 0.2 | 1:0.7 | formic acid (8.45), acetic acid (3.64), succinic acid (4.09) |

TABLE 6
Synergistic enhancement of the growth of E. faecium D, serotype 11
(DSMZ 20477) upon treatment with different concentrations of benzoic
acid (BA) in combination with different concentrations of organic acid.

| BA | OA | | |
|---|---|---|---|
| (mM) | (mM) | BA:OA | details |
| 20 | 100.0 | 1:5 | propionic acid (0.20) |
| 20 | 25.0 | 1:1.3 | propionic acid (0.33), succinic acid (0.18) |
| 20 | 12.5 | 1:0.6 | propionic acid (0.37) |
| 20 | 6.3 | 1:0.3 | acetic acid (0.18), propionic acid (0.18) |
| 20 | 3.1 | 1:0.2 | acetic acid (0.22), propionic acid (0.41), succinic acid (0.16) |
| 20 | 1.6 | 1:0.1 | acetic acid (0.38), propionic acid (0.50), succinic acid (0.43) |
| 20 | 0.8 | 1:0.04 | acetic acid (0.47), propionic acid (0.82), succinic acid (0.43) |
| 20 | 0.4 | 1:0.02 | acetic acid (0.62), propionic acid (0.77), succinic acid (0.25) |
| 20 | 0.2 | 1:0.01 | acetic acid (0.43) |
| 10 | 100.0 | 1:10 | propionic acid (0.22), succinic acid (0.67) |
| 10 | 50.0 | 1:5 | propionic acid (0.20), succinic acid (0.09) |
| 10 | 25.0 | 1:2.5 | acetic acid (1.04), propionic acid (0.01), fumaric acid (8.58), |
| | | | succinic acid (0.12) |
| 10 | 12.5 | 1:1.3 | acetic acid (3.06), propionic acid (1.59), fumaric acid (11.02), |
| | | | succinic acid (4.52) |
| 10 | 6.3 | 1:0.6 | formic acid (0.81), acetic acid (3.48), propionic acid (1.51), |
| | | | fumaric acid (5.12), succinic acid (11.79) |
| 10 | 3.1 | 1:0.3 | acetic acid (2.18), propionic acid (0.74), fumaric acid (0.28), |
| | | | succinic acid (12.94) |
| 5.0 | 100.0 | 1:20 | propionic acid (0.32), succinic acid (1.18) |
| 5.0 | 50.0 | 1:10 | formic acid (0.01), propionic acid (0.16), succinic acid (0.51) |
| 5.0 | 25.0 | 1:5 | formic acid (0.02), acetic acid (6.86), propionic acid (0.52), |
| | | | fumaric acid (19.81), succinic acid (0.23) |
| 5.0 | 12.5 | 1:2.5 | formic acid (0.74), acetic acid (13.29), propionic acid (15.73), |
| | | | succinic acid (12.56) |
| 5.0 | 6.3 | 1:1.3 | formic acid (6.38), succinic acid (18.21) |
| 5.0 | 3.1 | 1:0.6 | succinic acid (8.61) |
| 5.0 | 0.2 | 1:0.04 | succinic acid (0.05) |
| 2.5 | 100.0 | 1:40 | formic acid (0.06), propionic acid (0.32), succinic acid (1.43) |
| 2.5 | 50.0 | 1:20 | formic acid (0.23), propionic acid (0.32), succinic acid (0.72) |
| 2.5 | 25.0 | 1:10 | formic acid (0.32), acetic acid (7.78), propionic acid (0.61), |
| | | | fumaric acid (14.57), succinic acid (0.39) |
| 2.5 | 12.5 | 1:5 | formic acid (1.33), propionic acid (19.62), succinic acid (15.68) |
| 2.5 | 6.3 | 1:2.5 | succinic acid (12.51) |
| 2.5 | 3.1 | 1:1.2 | succinic acid (15.41) |
| 2.5 | 1.6 | 1:0.6 | succinic acid (11.58) |
| 2.5 | 0.8 | 1:0.3 | succinic acid (3.63) |
| 2.5 | 0.4 | 1:0.2 | succinic acid (0.14) |
| 2.5 | 0.2 | 1:0.1 | succinic acid (8.94) |
| 1.3 | 100.0 | 1:76.9 | propionic acid (0.22), succinic acid (1.25) |
| 1.3 | 50.0 | 1:38.5 | formic acid (0.18), propionic acid (0.47), fumaric acid (0.22), |
| | | | succinic acid (0.77) |
| 1.3 | 25.0 | 1:19.2 | formic acid (0.22), acetic acid (8.09), propionic acid (0.72), |
| | | | succinic acid (0.18) |
| 1.3 | 12.5 | 1:9.6 | formic acid (1.59), succinic acid (14.77) |
| 1.3 | 3.1 | 1:2.4 | succinic acid (11.93) |
| 1.3 | 1.6 | 1:1.2 | succinic acid (2.04) |
| 1.3 | 0.2 | 1:0.2 | succinic acid (1.79) |
| 0.6 | 100.0 | 1:166.7 | acetic acid (0.26), propionic acid (0.20), fumaric acid (0.14), |
| | | | succinic acid (1.73) |
| 0.6 | 50.0 | 1:83.3 | formic acid (0.31), acetic acid (0.17), propionic acid (0.34), |
| | | | fumaric acid (0.16), succinic acid (0.69) |
| 0.6 | 25.0 | 1:41.7 | formic acid (0.56), acetic acid (7.87), propionic acid (0.65), |
| | | | succinic acid (0.37) |
| 0.6 | 12.5 | 1:20.8 | formic acid (1.85), succinic acid (6.05) |
| 0.6 | 3.1 | 1:5.2 | succinic acid (6.96) |
| 0.3 | 100.0 | 1:333.3 | acetic acid (0.14), propionic acid (0.56), fumaric acid (0.32), |
| | | | succinic acid (0.44) |
| 0.3 | 25.0 | 1:83.3 | acetic acid (5.76), succinic acid (0.33) |
| 0.3 | 12.5 | 1:41.7 | formic acid (1.19), succinic acid (5.87) |

TABLE 7
Synergistic enhancement of the growth of B. subtilis strain Marburg
(DSMZ 10) upon treatment with different concentrations of benzoic acid
(BA) in combination with different concentrations of organic acid.

| BA | OA | | |
|---|---|---|---|
| (mM) | (mM) | BA:OA | details |
| 20 | 100.0 | 1:5 | acetic acid (0.88), propionic acid (0.75), succinic acid (0.78) |
| 20 | 50.0 | 1:2.5 | acetic acid (0.83), propionic acid (1.11), succinic acid (0.60) |
| 20 | 25.0 | 1:1.3 | propionic acid (0.94), succinic acid (0.64) |
| 20 | 12.5 | 1:0.6 | propionic acid (0.18) |
| 20 | 0.2 | 1:0.01 | acetic acid (0.20) |
| 10 | 100.0 | 1:10 | formic acid (0.29), acetic acid (0.41), propionic acid (0.80), |
| | | | succinic acid (1.37) |
| 10 | 50.0 | 1:5 | acetic acid (0.42), propionic acid (0.54), succinic acid (0.61) |
| 10 | 25.0 | 1:2.5 | acetic acid (0.31), propionic acid (0.13), fumaric acid (149.45) |
| 10 | 12.5 | 1:1.3 | propionic acid (0.05), fumaric acid (115.97), succinic acid (135.41) |
| 10 | 6.3 | 1:0.6 | fumaric acid (147.52), succinic acid (86.11) |
| 10 | 3.1 | 1:0.3 | fumaric acid (98.19), succinic acid (108.45) |
| 10 | 1.6 | 1:0.2 | fumaric acid (21.77) |
| 10 | 0.8 | 1:0.1 | fumaric acid (27.61) |
| 10 | 0.4 | 1:0.04 | fumaric acid (27.26) |
| 10 | 0.2 | 1:0.02 | fumaric acid (31.49) |
| 5.0 | 100.0 | 1:20 | formic acid (0.24), acetic acid (0.29), propionic acid (0.34), |
| | | | fumaric acid (0.05), succinic acid (0.82) |
| 5.0 | 50.0 | 1:10 | succinic acid (0.20) |
| 5.0 | 25.0 | 1:5 | formic acid (0.42), propionic acid (0.49), fumaric acid (85.13), |
| | | | succinic acid (0.29) |
| 5.0 | 12.5 | 1:2.5 | formic acid (21.61), acetic acid (93.44), propionic acid (22.16), |
| | | | succinic acid (89.94) |
| 5.0 | 6.3 | 1:1.3 | formic acid (20.52), succinic acid (58.91) |
| 5.0 | 0.4 | 1:0.1 | succinic acid (4.87) |
| 5.0 | 0.2 | 1:0.04 | succinic acid (9.98) |
| 2.5 | 100.0 | 1:40 | formic acid (0.06), propionic acid (0.01), fumaric acid (0.54), |
| | | | succinic acid (0.82) |
| 2.5 | 50.0 | 1:20 | formic acid (0.37), acetic acid (0.50), propionic acid (0.34), |
| | | | fumaric acid (0.09), succinic acid (0.72) |
| 2.5 | 25.0 | 1:10 | formic acid (0.49), acetic acid (0.19), propionic acid (0.34), |
| | | | fumaric acid (96.81), succinic acid (0.19) |
| 2.5 | 12.5 | 1:5 | succinic acid (91.62) |
| 2.5 | 1.6 | 1:0.6 | fumaric acid (12.29) |
| 2.5 | 0.8 | 1:0.3 | acetic acid (1.21), fumaric acid (10.50) |
| 2.5 | 0.4 | 1:0.2 | acetic acid (12.44), fumaric acid (19.91) |
| 2.5 | 0.2 | 1:0.1 | acetic acid (3.99), fumaric acid (15.75) |
| 1.3 | 100.0 | 1:76.9 | formic acid (0.22), acetic acid (0.14), fumaric acid (0.06), |
| | | | succinic acid (0.61) |
| 1.3 | 50.0 | 1:38.5 | formic acid (0.18), propionic acid (0.15), succinic acid (0.77) |
| 1.3 | 25.0 | 1:19.2 | formic acid (0.30), succinic acid (0.31) |
| 1.3 | 12.5 | 1:9.6 | succinic acid (102.47) |
| 1.3 | 6.3 | 1:4.8 | fumaric acid (7.04) |
| 1.3 | 3.1 | 1:2.4 | fumaric acid (3.20) |
| 1.3 | 1.6 | 1:1.2 | acetic acid (27.40), fumaric acid (5.36) |
| 1.3 | 0.8 | 1:0.6 | acetic acid (21.47) |
| 1.3 | 0.4 | 1:0.3 | acetic acid (29.69), fumaric acid (5.52) |
| 1.3 | 0.2 | 1:0.2 | acetic acid (24.60) |
| 0.6 | 100.0 | 1:166.7 | formic acid (0.53), acetic acid (0.34), propionic acid (0.28), |
| | | | fumaric acid (0.99), succinic acid (1.04) |
| 0.6 | 50.0 | 1:83.3 | formic acid (0.46), acetic acid (0.17), propionic acid (0.35), |
| | | | succinic acid (0.81) |
| 0.6 | 25.0 | 1:41.7 | formic acid (0.44), succinic acid (0.30) |
| 0.6 | 12.5 | 1:20.8 | formic acid (0.94), acetic acid (15.04), fumaric acid (6.93), |
| | | | succinic acid (14.32) |
| 0.6 | 6.3 | 1:10.5 | formic acid (19.07), fumaric acid (3.89) |
| 0.6 | 3.1 | 1:5.2 | acetic acid (16.30) |
| 0.6 | 1.6 | 1:2.7 | acetic acid (21.41) |
| 0.6 | 0.8 | 1:1.3 | acetic acid (24.80) |
| 0.6 | 0.4 | 1:0.7 | acetic acid (37.11) |
| 0.6 | 0.2 | 1:0.3 | acetic acid (30.45) |
| 0.3 | 100.0 | 1:333.3 | formic acid (0.51), acetic acid (0.04), fumaric acid (0.83), |
| | | | succinic acid (0.68) |
| 0.3 | 50.0 | 1:166.7 | formic acid (0.12), succinic acid (0.34) |
| 0.3 | 25.0 | 1:83.3 | formic acid (0.41), succinic acid (0.27) |
| 0.3 | 12.5 | 1:41.7 | formic acid (26.34), acetic acid (0.47) |
| 0.3 | 6.3 | 1:21 | formic acid (0.66) |
| 0.3 | 3.1 | 1:10.3 | acetic acid (11.84) |
| 0.3 | 1.6 | 1:5.3 | acetic acid (24.66) |
| 0.3 | 0.8 | 1:2.7 | acetic acid (20.52) |
| 0.3 | 0.4 | 1:1.3 | acetic acid (34.92) |
| 0.3 | 0.2 | 1:0.7 | acetic acid (19.33) |

1. A composition for modulating growth of microorganisms, wherein the composition comprises a) benzoic acid and/or at least one salt thereof, and b) at least one additional organic acid and/or at least one salt thereof, wherein the composition modulates the growth of microorganisms in a synergistic model.

2. The composition of claim 1, wherein the at least one additional organic acid is selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and/or keto carboxylic acids.

3. The composition of claim 1, wherein the at least one additional organic acid is selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic acid, decanoic acid, dodecanoic (lauric) acid, lactic acid, malic acid, citric acid, tartaric acid, cinnamic acid, pyruvic acid, gluconic acid, suberic acid, malonic acid, tannic acid, caffeic acid, ellagic acid, perillic acid and gallic acid.

4. The composition of claim 1, wherein the at least one additional organic acid is selected from the group consisting of formic acid, acetic acid, propionic acid, fumaric acid and succinic acid, preferably fumaric acid and succinic acid.

5. The composition claim 1, wherein the at least one salt of the organic acid is any one of metal salts such as potassium, sodium and calcium salts, and ammonium salts.

6. The composition of claim 1, wherein the at least one salt of benzoic acid is selected from the group consisting of sodium benzoate, magnesium benzoate, manganese benzoate, potassium hypophosphite, aluminium benzoate, calcium benzoate and ferric benzoate.

7. The composition of claim 1, wherein the molar ratio of the at least one additional organic acid and/or the at least one salt thereof to benzoic acid and/or the at least one salt thereof is 0.001:1 to 500:1, preferably from 0.005:1 to 300:1, more preferably from 0.01:1 to 200:1, such as from 0.01:1 to 0.15:1, from 0.02:1 to 0.1:1, from 0.04:1 to 100:1, from 1:1 to 100:1, from 10:1 to 80:1 and from 20:1 to 40:1.

8. The composition of claim 1, wherein the at least one additional organic acid and/or the at least one salt thereof is provided at any concentration from 0.2 mmol to 100 mmol, in combination with benzoic acid and/or at least one salt thereof at any concentration from 0.2 mmol to 20 mmol, per 1 Kg of the composition in solid form or per 1 L of the composition in liquid form.

9. The composition of claim 1, wherein the composition comprises benzoic acid and/or at least one salt thereof and the at least one additional organic acid and/or at least one salt thereof as the only active ingredients for modulating growth of microorganisms.

10. The composition of claim 1, wherein the microorganisms is any one or more of the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, Lactobacillus, Bifidobacterium, Saccharomyces, Bacillus, Pediococcus, Enterococcus, Streptococcus, Propionibacterium, Pseudomonas and Citrobacter; preferably is selected from the group consisting of Escherichia coli, Salmonella enterica, Shigella sonnei, Clostridium perfringens, Lactobacillus reuteri, Enterococcus faecium and Bacillus subtilis.

11. Use of the composition according to claim 1 for modulating growth of microorganisms.

12. Use of the composition according to claim 1 for modulating growth of microorganisms in the gastrointestinal tract of animals, and/or for improving gut health of animals, and/or for the treatment, amelioration and/or prevention of post-weaning diarrhea (PWD), and/or improving growth performance of animals.

13. Use of claim 11, wherein the animal is selected from the group consisting of sheep, goats, cattle, pigs (including but not limited to piglets, growing pigs, and sows), poultry (including but not limited to turkeys, ducks, quail, guinea fowl, geese, pigeons and chicken) and horses.

14. Use of at least one additional organic acid and/or at least one salt thereof in the composition according to claim 1 for boosting the effect of benzoic acid and/or at least one salt thereof in modulating growth of microorganisms.

15. A method for modulating growth of microorganisms, wherein the method comprises the steps of a) providing the composition according to claim 1; and b) contacting the composition of a) with the microorganisms.

16. A method for modulating growth of microorganisms in the gastrointestinal tracts of animals, and/or for improving gut health of animals, and/or for the treatment, amelioration and/or prevention of post-weaning diarrhea (PWD), improving growth performance of animals, wherein the method comprises the steps of a) providing the composition according to claim 1; and b) administering to the animals the composition of a).

17. A method for boosting the effect of benzoic acid and/or at least one salt thereof in modulating growth of microorganisms, wherein the method comprises: a) providing the composition according to claim 1; and b) contacting the composition of a) with the microorganisms.

18. A method for boosting the effect of benzoic acid and/or at least one salt thereof in modulating growth of microorganisms in the gastrointestinal tracts of animals, and/or in improving gut health of animals, and/or in the treatment, amelioration and/or prevention of post-weaning diarrhea (PWD) of animals, wherein the method comprises the steps of a) providing the composition according to claim 1; and b) administering to the animals the composition of a).
